(19) United States
(12) Patent Application Publication
(10) Pub. No.: US 2007/0043412 A1
(43) Pub. Date: Feb. 22, 2007

(54) Cardiac electrode assembly

(76) Inventors: Herve Janssens, Gent (BE); Robert G. Walsh, Lakeville, MN (US); Paul Andrew Pignato, Stacy, MN (US); Ann Margaret Thomas, Plymouth, MN (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 11/207,682

(22) Filed: Aug. 18, 2005

(51) Int. Cl. A61N 1/05

(52) U.S. Cl. 607/119

(57) ABSTRACT

A cardiac electrode assembly includes an electrode lead with a plurality of electrical conductors in a common lead jacket. The electrical conductors include a primary conductor and at least a first secondary conductor. At a proximal end of the lead, the conductors terminate at a connector having a plurality of exposed electrical contacts. The contacts include a primary contact connected to the primary conductor and a secondary contact connected to the secondary conductor. A plurality of cardiac electrodes is mechanically connected to the distal end of the lead. The plurality includes a primary cardiac electrode and at least a secondary cardiac electrode (more preferably, at least two secondary cardiac electrodes) connected to one or more secondary conductors. In still preferred embodiments of the invention, multiple secondary conductors with separate leads in the common jacket are connected to the distal end of the common lead jacket.

### I. BACKGROUND OF THE INVENTION

1. Field of the Invention

This invention pertains to a cardiac electrode assembly for pacing, sensing or applying signals to a tissue of a heart.

2. Description of the Prior Art

Cardiac electrodes have long been used on the heart for sensing electrical activity on the heart and for the treatment of a variety of disorders including cardiac asynchrony. Cardiac electrodes come in many varieties of shapes and sizes and configurations including a wide variety of features for securing the electrode to a tissue of the heart. For example, such electrodes may include so-called screw-in type or “pigtail” electrodes for burrowing into the tissue of the heart to secure the electrode on the heart. Also, electrodes can be urged against the heart to make contact with the heart surface. Such electrodes may be individually placed on the heart. Japanese Pat. No. 2271829 dated November 1990 shows electrodes for diagnosis of infarction. The electrodes are attached to a net temporarily surrounding the heart.

Cardiac electrodes may be placed percutaneously or surgically. A percutaneous placement includes advancing an electrode through the vasculature of the patient into a chamber of the heart and then placing the electrode in residence within the chamber of the heart. Percutaneous placement may also include placing an electrode near the epicardial surface of the heart by advancing the electrode into a coronary vessel near the epicardial surface.

A surgical placement includes surgically accessing the epicardial surface of the heart and placing electrodes on or near the epicardial surface. The surgical access may include minimally invasive surgical techniques.

Percutaneous delivery of electrodes has certain desirable features. For example, such a procedure is normally regarded as less invasive than a surgical access.

Notwithstanding advantages, percutaneous delivery of cardiac electrodes has limitations. For example, a percutaneous delivery for epicardial stimulation requires advancement of electrodes and their associated leads through the coronary vasculature of the patient. Such vasculature has a narrow diameter and often presents a tortuous path limiting the ability to place such electrodes. Further, even if an electrode can be advanced into the coronary vasculature, only a very limited surface area of the epicardium of the heart can be treated in this manner. Percutaneously accessible blood vessels may not be overlying the most desirable area of the heart for treatment. In contrast, a surgical delivery permits placement of an electrode at any location on the epicardium of the heart.

Pacing electrodes are typically driven by direct current (DC) voltage systems from an implantable pulse generator or other power source. Pacing electrodes are commonly either uni-polar or bi-polar.

A uni-polar electrode has a single contact near the tissue to be treated. Current flow from the electrode (normally positively charged) passes through tissue to a more remote electrical ground or oppositely polarized electrode (e.g., an exposed ground or negatively charged electrode on the implantable pulse generator).

A bi-polar electrode includes two oppositely charged electrodes to create a more focused and localized field of current flow through the target tissue. As a result, a bi-polar electrode assembly includes a pair of electrodes for any given treatment with an associated positive-voltage electrode coupled with an associated negative-voltage electrode.

Paired electrodes may have separate leads (conductors contained within flexible, bio-compatible, electrically insulating jackets) or the paired electrodes may have a common lead. An associated pair of electrodes with a common lead is the CapSure® Epi lead of Medtronic Inc., Minneapolis, Minn., U.S.A.

In the CapSure® Epi lead, a positive and a negative pacing electrode with separate flexible leads are connected to a common hub with a common lead extending from the hub to a connector. The connector can then be connected to an implantable pulse generator or other source of a pacing signal.

Paired electrodes such as the CapSure® Epi electrode assembly also have certain limitations. Where it is desirable to provide pacing over a wide surface area or at multiple locations on the heart, multiple electrode assemblies are required each with individual pairs of differently polarized electrodes creating separate fields for pacing. Accordingly, if three different areas are to be paced, six electrodes must be placed on the heart. Also, over time the desired location for optimized pacing may change. A previously placed electrode may no longer be in optimal location and the patient must either cope with sub-optimal pacing or endure a subsequent procedure for re-positioning of electrodes.

### II. SUMMARY OF THE INVENTION

According to a preferred embodiment of the present invention, a cardiac electrode assembly is disclosed having an electrode lead with a plurality of electrical conductors in a common lead jacket. The electrical conductors include a primary conductor and at least a first secondary conductor. At a proximal end of the lead, the conductors terminate at a connector having a plurality of exposed electrical contacts. The contacts include a primary contact connected to the primary conductor and a secondary contact connected to the secondary conductor. A plurality of cardiac electrodes is mechanically connected to the distal end of the lead. The plurality includes a primary cardiac electrode and at least a secondary cardiac electrode (more preferably, at least two secondary cardiac electrodes) connected to one or more secondary conductors. In still preferred embodiments of the invention, multiple secondary conductors with separate leads in the common jacket are connected to the distal end of the common lead jacket.

### III. BRIEF DESCRIPTION OF THE DRAWINGS

FIG. 1 is a plan view of a cardiac electrode assembly according to the present invention;

FIG. 1A is a view of an alternative embodiment of a connector for the electrode assembly of FIG. 1;

FIG. 2 is an electrical schematic representation of the electrical components of the electrode assembly of FIG. 1;

FIG. 2A is an electrical schematic representation of the electrical components of the electrode assembly of FIG. 1 adapted with the connector of FIG. 1A;

FIG. 3 is a schematic presentation of the electrode assembly of FIG. 1 operatively positioned on the epicardial surface of a patient's heart;

FIG. 4 is a cross sectional view of a heart showing the electrode assembly of FIG. 1 placed on the heart with an alternative placement having at least one of the electrodes of FIG. 1 imbedded within the tissue of the heart;

FIG. 5 is a table showing alternative polarization of the electrodes of the electrode assembly of FIG. 1;

FIG. 6 is a schematic representation of an external controller having wireless transmission to an implanted member for pacing the electrodes of FIG. 1 in an alternative embodiment;

FIG. 7 is a schematic representation of a wireless control for independently controlling each of the independent electrodes on a surface of a heart;

FIG. 8 is a schematic representation of the circuitry of the electrode assembly of FIG. 1 adapted to provide time delay between the energizing of secondary electrodes.

FIG. 9 illustrates placement of electrodes on a carrier surrounding a heart;

FIG. 10 illustrates placement of an array of electrodes on a carrier surrounding a heart;

FIG. 11 illustrates the array of electrodes of FIG. 10 in a row and column format;

FIG. 12 shows the electrode array of FIG. 11 electrically connected to a controller;

FIG. 13A illustrates the electrodes of FIG. 1 energized with a field between electrode pairs E₁, E₂;

FIG. 13B illustrates the electrodes of FIG. 12A with the fields shifted between electrodes E₁, E₃; and

FIG. 13C is a view of FIG. 12A with a field shifted between electrode pairs E₁, E₄.

### IV. DETAILED DESCRIPTION OF THE PREFERRED EMBODIMENT

With reference now to the various drawing figures in which identical elements are numbered identically throughout, a description of a preferred embodiment of the present invention will now be provided. The following patents and published applications, described elsewhere in this application, are incorporated herein by reference: U.S. Pat. No. 6,907,285 issued Jun. 14, 2005; U.S. Pat. No. 6,893,392 issued May 17, 2005; U.S. Pat. No. 5,702,343 issued Dec. 30, 1997; U.S. Pat. No. 6,123,662 issued Sep. 26, 2000; U.S. Pat. No. 6,482,146 issued Nov. 19, 2002; U.S. Pat. No. 6,730,016 issued May 4, 2004; U.S. Pat. No. 6,425,856 issued Jul. 30, 2002; U.S. Pat. No. 6,572,533 issued Jun. 3, 2003; and U.S. patent application Ser. No. 10/165,504 filed Jun. 7, 2002 and published Dec. 12, 2003 as Publication No. 2003-0229265A1.

Novel Electrode Assembly Design for IS-1 Connector

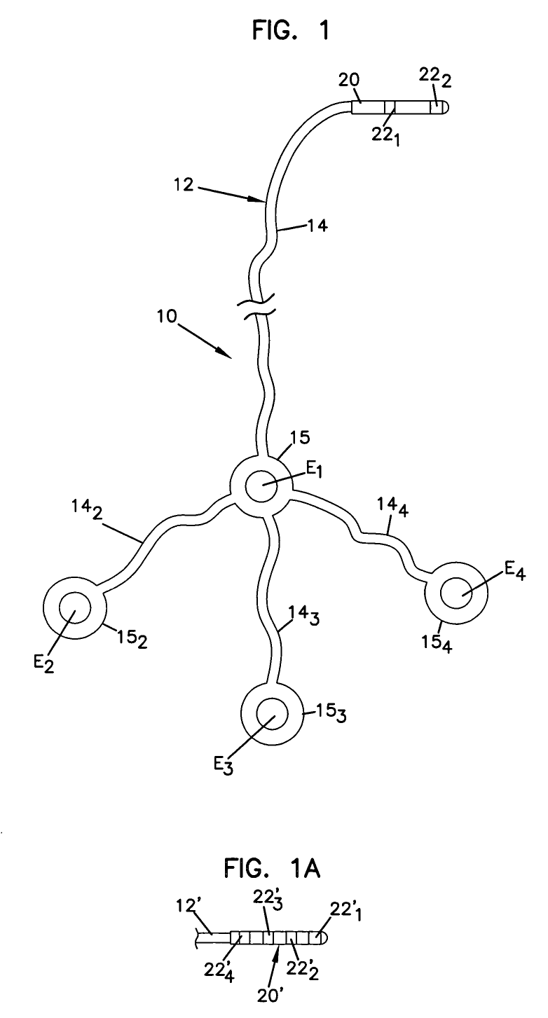

Referring first to FIG. 1, an electrode assembly 10 according to the present invention includes a plurality of electrodes. This plurality of electrodes includes a primary electrode E₁ and, in the embodiment of FIG. 1, three secondary electrodes E₂, E₃, and E₄. It is preferred that at least two secondary electrodes (for example E₂ and E₃) be provided with a single primary electrode E₁ for reasons that will become apparent.

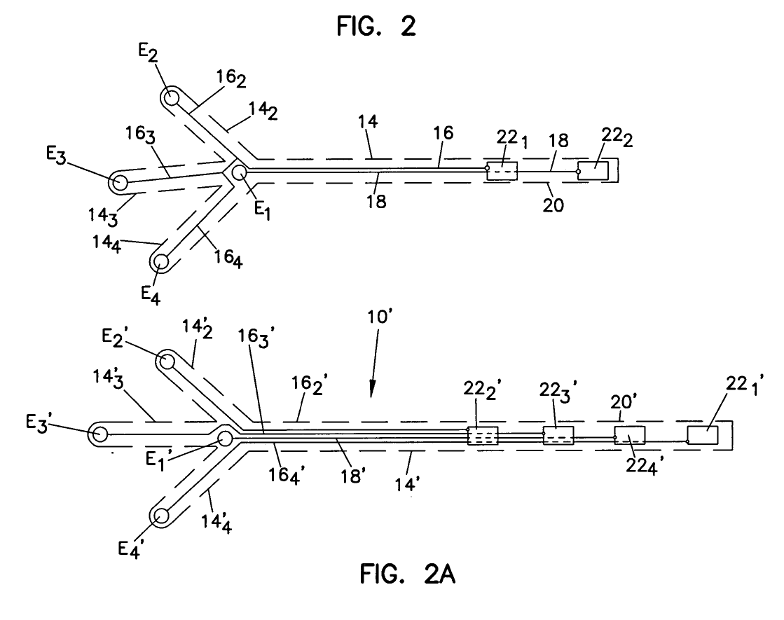

In the embodiment of FIG. 1, the electrodes E₁ through E₄ have a common main lead 12. The main lead 12 includes a common lead jacket 14 covering a plurality of conductors. For the embodiment of FIG. 1, two such lead conductors 16, 18 are carried within the jacket 14 as shown in FIG. 2. Conductor 18 is a primary conductor connected to primary electrode E₁. Conductor 16 is a secondary conductor conducted in parallel to each of secondary electrodes E₂, E₃, and E₄ by branch conductors 16₂, 16₃, and 16₄ respectively.

Each of conductors 16₂, 16₃, and 16₄ is sheathed in a highly flexible jacket 14₂, 14₃, and 14₄, which extend from a jacket hub 15 surrounding primary electrode E₁. The material of jackets 14 and 14₂ through 14₄ (shown in phantom lines in FIG. 2) may be any highly flexible, polyurethane, biocompatible, electrically insulating material such as silicone or the like. A hub 15₂, 15₃, and 15₄ surrounds each of the electrodes E₂, E₃, and E₄. The electrodes E₁ through E₄ are exposed through their respective hubs. The hubs 15, 15₂, 15₃, and 15₄ may be the same material (and simultaneously molded) as the jackets 14 and 14₂ through 14₄.

At a proximal end of the lead 12, a connector 20 is secured to the lead 12. In the embodiment of FIGS. 1 and 2, the connector 20 is a so-called IS-1 connector which is a conventional connector having two exposed electrical contacts 22₁ and 22₂. Other conventional connectors include VS-1 and LV-1 connectors known in the art. Contact 22₂ is a primary contact connected to electrode E₁ by direct connection of the contact 22₂ to conductor 18. Contact 22₁ is a secondary contact connected to conductor 16 and, hence, to each of electrodes E₂ through E₄.

Connectors such as the IS-1 connector 20 are well known and form no part of this invention per se. Such connectors are used for connection of cardiac electrode assemblies to implantable pulse generators as is known in the art.

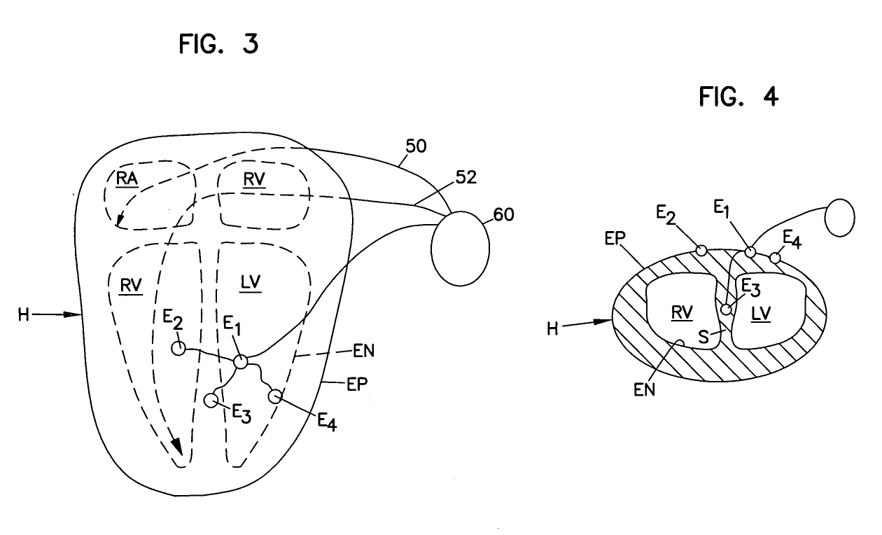

With the embodiment of FIG. 1, the electrode assembly 10 may be placed on a patient's heart with the electrodes E₁ through E₄ directly placed in electrically conducting contact with the epicardial tissue EP of the heart H as illustrated in FIG. 3. In FIG. 3, traditional percutaneously delivered prior art electrode assemblies 50, 52 are shown in phantom lines in residence within the right atrium RA and right ventricle RV and near the endocardium EN of the heart H as is conventional. It is anticipated that the present invention may be used either alone or in combination with such prior art electrode assemblies for percutaneously delivered electrodes.

The electrode assembly 10 of the present invention is placed with the electrodes E₁ through E₄ on the heart H. While the electrodes E₁ through E₄ are illustrated as exposed contacts (which make electrical connection by being urged against the heart), it will be appreciated that the electrodes E₁ through E₄ can take any configuration known in the art including so-called pigtail electrodes, which may have a portion which is imbedded within the heart tissue. Furthermore, through use of a needle placement or the like, one or more of the electrodes E₁ through E₄ may be fully imbedded within the tissue of the heart H. This is illustrated in FIG. 4 where electrode E₃ is shown imbedded within the septal wall S of the heart H.

The electrode assembly 10 is connected to an implantable pulse generator 60 by the connector 20 inserted within a mating connector (not shown) on the implantable pulse generator 60 as is conventional for attachment of prior art electrode assemblies 50, 52. The implantable pulse generator 60 provides a signal to the conductors 16, 18 such that the primary electrode E₁ may have a polarity indicating a positive charge and the electrodes E₂ through E₄ may be simultaneously negatively charged. In addition to application of a signal to the heart, the electrodes E₂ through E₄ may be used as sensing electrodes for delivering electrical signals from the heart H to monitoring or diagnostic equipment.

As a result of the placement thus described, the implantable pulse generator 60 may generate a positive polarity on contact 222 and a negative polarity on contact 221. As a result, three different electrical fields are created extending between the electrode pairs E₁, E₂; E₁, E₃ and E₁, E₄. As a result, three different pacing areas are provided with four electrodes where the prior art would require six such electrodes being placed on the heart.

Also, the prior art electrode assemblies (such as the CapSure® Epi electrode assembly) would require three pacing leads connected to a pulse generator to create three electrical fields on the heart. The present invention utilizes a single pacing lead 14. Since pulse generators have only a limited number of connector locations, a more effective pacing therapy is possible with the present invention.

A surgeon placing the electrode assembly 10 on the heart may place the primary electrode E₁ in any desired location and extend the secondary electrodes E₂ through E₄ to any one of a number of desired locations on the heart limited only by the length of the secondary jackets E₂ through E₄. It is anticipated that a representative length of a secondary jacket 14₄ would be about 1 to 3 centimeters.

Variable Timing of Energizing Electrode Pairs

With the invention thus described, the secondary electrodes E₂ through E₄ all receive a negative charge during the pacing at the same instance as each of the other secondary electrodes E₂ through E₄. It may be desirable for the electrodes E₂ through E₄ be provided with a charge at different times to create a wave of paced tissue along the surface of the heart.

For example, and as illustrated in FIGS. 14A-14C, it may be desirable to have a field F₄ between electrodes E₁, E₄ (FIG. 14C) at a time when no charge is provided to electrodes E₂ and E₃. Subsequently, it may be desirable to terminate the charge to electrode E₄ and provide a charge in electrode E₃ (to create field F₃ shown in FIG. 14B) for a limited period of time followed by a charge in electrode E₂ (to create field F₂ shown in FIG. 14A). Such a variation in timing of the application of a charge to electrodes E₂ through E₄ may create a wave effect of applying the pacing around the primary electrode E₁.

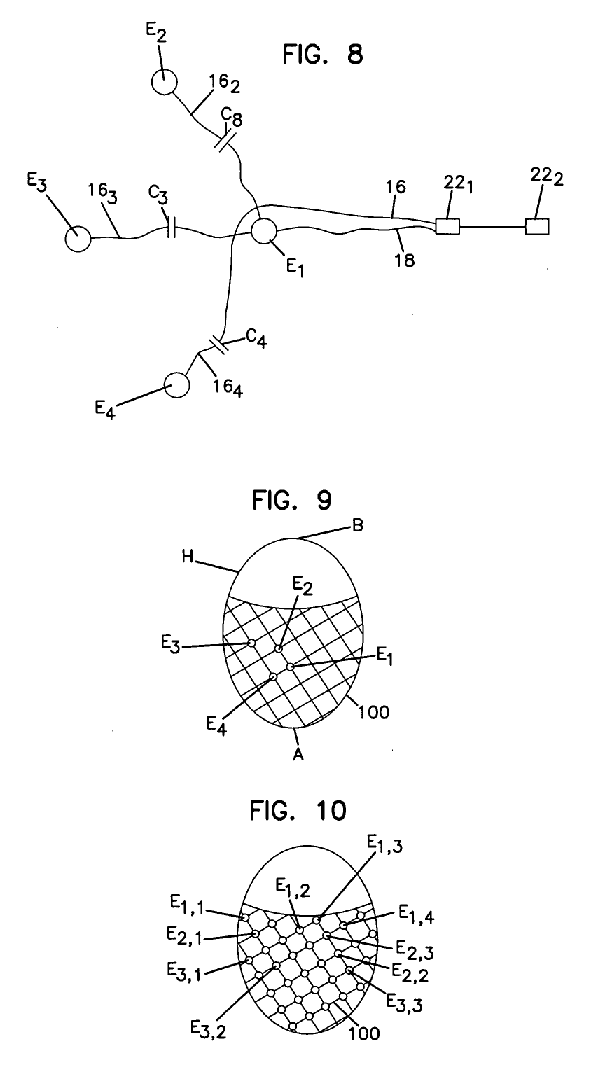

Altering the timing of energizing the secondary electrodes may be accomplished by providing capacitors C₂, C₃ and C₄ on each of secondary conductors 16₂ through 16₄ as illustrated in FIG. 8. Each of the capacitors C₂ through C₄ has a different capacitance to affect a different timing of application of the charge to the electrodes E₂, E₄. It will be appreciated that the use of a capacitance to cause a time delay in the charge application between the secondary electrodes E₂-E₄ as illustrated in FIG. 8 is representative of only one possible mechanism for providing a time delay on the charges.

Wireless Signal Transmission

As illustrated in FIG. 3, the electrode assembly 10 is connected to an implantable pulse generator 60 which may contain a battery and other power source as well as logic circuits for controlling the application of the pacing signal to the electrode assembly 10 as is conventional with respect to application of the pacing signals to electrode assemblies 50, 52. However, it is known in the art for a pacing signal to come from an external source such as an external pulse generator which is coupled to an implanted antenna or the like for the delivery of the pacing signals to the electrodes E₁ through E₄. For example, electrodes on separate PTFE arms for placement on opposite sides of phrenic nerve for quad-polar stimulation are described in a product brochure “ATROSTIM Phrenic Nerve Stimulator”, AtroTech Oy, P.O. Box 28, FIN-33721 Tampere, Finland (June 2004). The ATROSTIM sends signals from an external controller to an implanted antenna.

The explanted source of the pacing signal is illustrated as 70 in FIG. 6 connected by a wireless transmission path (such as a radio frequency transmission path 80) to an implanted antenna or other receiving member 50 hard-wired to the electrodes E₁ through E₄. Alternatively, each of the electrodes E₁ through E₄ may be independent members which receive separate pacing signals 80₁-80₄ directly through RF transmission from a transmitting controller 70′ (either implanted or external) as illustrated in FIG. 7. In FIG. 7, each of the electrodes E₁ through E₄ contains a receiving circuit for receiving the signal and creating a pacing in response to the received signal. While the wireless transmission is described with reference to sending pacing signals to electrodes, it is also applicable to sending sensed signals from electrodes. Wireless transmission from a controller to an implanted electrode is shown in U.S. Pat. No. 6,907,285 to Denker, et al., dated Jun. 14, 2005.

Novel Electrode Assembly Design for IS-4 Connector

FIGS. 1 and 2 illustrate an embodiment with each of the secondary electrodes E₂ through E₄ connected across a common conductor 16 to a contact 22, on the two-contact IS-1 connector. Presently, so-called IS-4 connectors are in development, which contain four electrical contacts for connection to an implantable pulse generator or the like.

Such an IS-4 connector is schematically illustrated as connector 20′ in FIG. 1A. FIG. 2A illustrates the IS-4 connector 20′ connected to a modified electrode assembly 10′. Elements in common between assemblies 10, 10′ are similarly numbered with the addition of an apostrophe to distinguish the embodiments. Except necessary to explain differences, similar elements are not separately described.

The connector 20′ contains contacts 22₁′ through 22₄′ individually connected to separate conductors including a primary conductor 18′ connecting primary electrode E₁′ to contact 22₁′. Secondary conductors 16₂′ through 16₄′ connect electrodes E₂′ through E₄′, respectively, to electrodes 22₂′ through 22₄′.

Each of the electrodes E₂′ through E₄′ may be independently controlled. The independent control may include a time delay control achieving the benefits associated with FIGS. 14A-14C or independent output, pulse with or sensing settings for each electrode depending on the individual thresholds to optimize the battery consumption and efficacy.

Each of the electrodes E₂′ through E₄′ may be controlled so that a secondary electrode E₂′ through E₄′ is dormant for an extended period of time. Namely, from time to time, the location of a desired pacing site may change for a particular patient. After the assembly 10′ is initially placed, the most desirable pacing location may be identified as the pacing pair E₁′ and E₄′. Accordingly, electrodes E₂′ and E₃′ may be left dormant. Over time, for a particular patient, it may be determined that the most desirable pacing location has a shift to the pacing pair E₁′ and E₂′. As a result, internal circuitry within the implanted controller may be adjusted so that only that pair E₁′, E₂′ is now energized and the remaining secondary electrodes E₃′ and E₄′ are dormant.

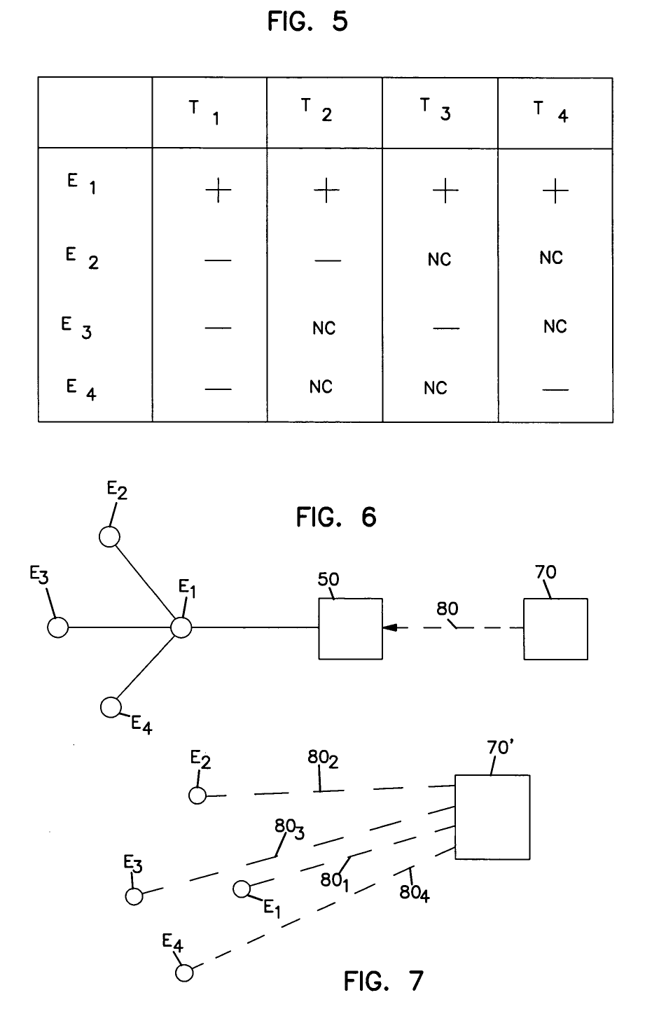

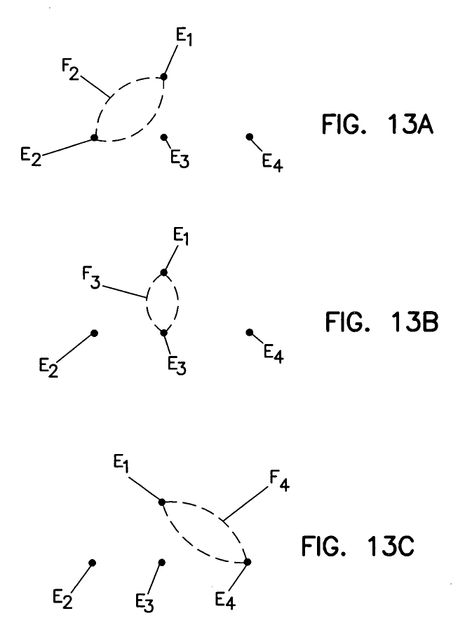

FIG. 5 illustrates options for controlling pacing pairs over time indicating that at times T₁, electrode E₁ is charged with a positive charge with electrodes E₂ through E₄ all simultaneously negatively charged. This would be the charge configuration associated with FIG. 1 without the benefit of the capacitors of FIG. 8. However, it may be desirable to alter the pacing such that at a time T₂ only electrode E₂ has a negative charge coupling the electrode E₂ to create a field (such as field F₂ in FIG. 13A) with electrode E₁. Electrodes E₃ and E₄ may be left dormant indicated by NC (no charge) in FIG. 5. At time T₃, electrode E₃ is charged and secondary electrodes E₂, E₄ have no charge (creating the field F₃ of FIG. 13B). At subsequent time T₄, electrode E₄ is negatively charged and electrodes E₂ and E₃ have no charge (creating the field F₄ of FIG. 13C). Accordingly from T₂ through T₄ a wave of paced tissue is created along the surface of the heart.

Placement of Electrodes with a Carrier

While the electrodes E₁ through E₄ are most conveniently placed on the common lead 12 as illustrated in FIG. 1, the electrodes E₁ through E₄ may be placed on an article surrounding the heart. For example, FIG. 9 shows a heart H having a jacket 100 surrounding the heart. The electrodes E₁ through E₄ are placed on the jacket. Alternative to a jacket 100 surrounding the heart H, the electrodes could be placed on a patch covering only a portion of the heart. Such a patch is shown in commonly assigned U.S. Pat. No. 6,893,392 issued May 17, 2005.

While the jacket 100 could be non-therapeutic independently, the jacket 100 is preferably a device selected to provide a therapeutic benefit such as a device for treating congestive heart failure as disclosed in Assignee's U.S. Pat. No. 5,702,343 issued Dec. 30, 1997; U.S. Pat. No. 6,123,662 issued Sep. 26, 2000 and U.S. Pat. No. 6,482,146 issued Nov. 19, 2002. These patents describe a technique for treating congestive heart failure by placing a cardiac support device in the form of a jacket around the heart. In certain of the specific embodiments disclosed, the jacket is a knit of polyester material which surrounds the heart and which provides resistance to progressive diastolic expansion. Other described materials include metal such as stainless steel (the jacket of the present invention may also be made in whole or part of nitinol). In certain aspects, the knit side and open cell size are selected to minimize or control fibrosis. It is believed that such resistance decreases wall tension on the heart and permits a diseased heart to beneficially remodel.

Assignee's U.S. Pat. No. 6,730,016 issued May 4, 2004 describes a jacket with a non-adherent lining or coating. In certain embodiments, the coating is in specific locations (for example, over surface-lined cardiac blood vessels). Assignee's U.S. Pat. No. 6,425,856 issued Jul. 30, 2002 describes a cardiac jacket with therapeutic agents incorporated on the jacket for providing additional therapy to the heart. Assignee's U.S. Pat. No. 6,572,533 issued Jun. 3, 2003 describes a treatment on the left ventricle side of the heart only. Assignee's U.S. patent application Ser. No. 10/165,504 filed Jun. 7, 2002 and published Dec. 12, 2003 as Publication No. 2003-0229265A1 teaches a highly compliant cardiac jacket.

In FIG. 9, the jacket 100 is shown covering the apex A of the heart H but not covering the atria near the base B of the heart H. It will be appreciated that the jacket or a portion thereof could cover the atria of the heart.

The electrodes E₁ through E₄ may be fixed to the jacket 100 at manufacture of the 100 jacket or may conveniently be attached to the jacket or the heart in the region of the jacket following placement of the jacket 100 on the heart.

Multiple Redundant Electrodes

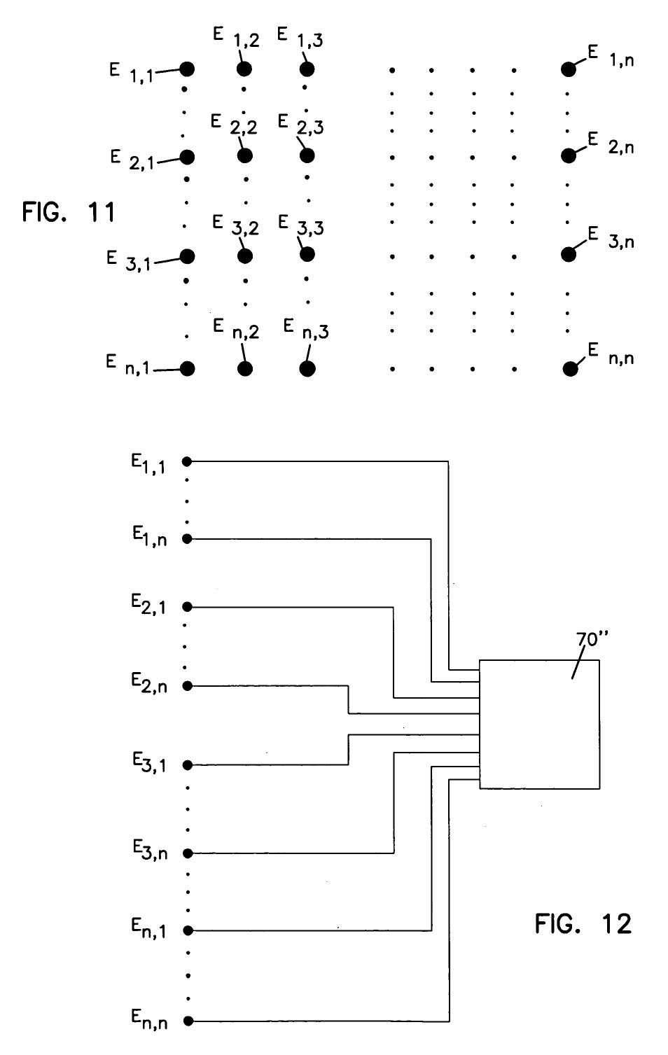

FIG. 10 illustrates an array of electrodes placed on the jacket. The array is shown flat in FIG. 11 as including a row of electrodes E₁,₁ through E₁,ₙ, a second row of electrodes E₂,₁ through E₂,ₙ, a third row of electrodes E_{3,1} through E₃,ₙ and continuing to an n-th row of electrodes Eₙ,₁ through E_{n,n}.

Each of the electrodes of the array may be connected to a controller as previously described which may be fully implantable or may be activated through RF or other wireless transmission. Further, the electrodes may also be coupled to a controller (again, either hardwired or through wireless transmission) to provide sensing signals to the controller.

The size of the array is selected so that the number of electrodes is in excess of the number otherwise desired for providing sensing or pacing functions on the heart. Preferably, the electrodes of the array are secured to the fabric of the jacket 100 at time of manufacture and before placement on the heart.

As a result of the excess number of electrodes, there is a redundancy in the number of electrodes such that at the time of placing the jacket 100 on the heart, a surgeon need not be concerned with precise placement of any given electrode over any given location on the heart. Instead, after placement of the electrode jacket 100 on the heart, the electrodes of the array may be individually sensed for determining which of the electrodes is most preferably energized for optimizing a pacing function on the heart. Those electrodes may be energized by internal switches within the controller 70″. The remaining electrodes may be left dormant.

Over time, the location on the heart for optimized pacing may change. As a result, a patient may have all the electrodes of the array interrogated to sense and determine locations on the heart through which a pacing would be most beneficial. In the event such optimal locations have changed over time, the originally paced electrodes may be shifted to a dormant state and the newly identified optimal electrodes may be shifted from a dormant state to a paced state by the controller 70″.

It has been shown how the objects of the present invention have been achieved in a preferred embodiment. Modifications and equivalents of the disclosed concepts are intended to be included within the scope of the claims, which are appended hereto.

We claim:

1. A cardiac electrode assembly comprising:
an electrode lead including a plurality of electrical conductors in a common lead jacket, said at least two conductors including a primary conductor and at least a first secondary conductor;
at a proximal end of said lead, said conductors terminating at a connector having a plurality of exposed electrical contacts, said contacts including a primary contact and at least a first secondary contact;
said primary contact connected to said primary conductor and said first secondary contact connected to said secondary conductor;
a plurality of cardiac electrodes mechanically connected to a distal end of said lead, said plurality including at primary cardiac electrode and at least a first and a second secondary cardiac electrode;
said primary cardiac electrode connected to said primary conductor; and
at least said first secondary cardiac electrode connected to said first secondary conductor.

2. A cardiac electrode assembly according to claim 1 wherein said second secondary cardiac electrode is connected to said first secondary conductor.

3. A cardiac electrode assembly according to claim 1 further comprising:
a source of a pacing signal including a primary polarity signal and a first secondary polarity signal;
said primary polarity signal connected to said primary contact and said first secondary signal connected to said first secondary contact.

4. A cardiac electrode assembly according to claim 3 wherein said source of a pacing signal is a pulse generator connected to said contacts by electrical conductors.

5. A cardiac electrode assembly according to claim 3 wherein said source of a pacing signal is a pulse generator connected to said contacts by wireless transmission.

6. A cardiac electrode assembly according to claim 1 further comprising:
said plurality of electrical conductors including at least a second secondary conductor;
said plurality of exposed electrical contacts including at least a second secondary contact, said second secondary contact connected to said second secondary conductor;
said second secondary cardiac electrode is connected to said second secondary conductor.

7. A cardiac electrode assembly according to claim 6 further comprising:
a source of a pacing signal including a primary polarity signal, a first secondary polarity signal and a second secondary polarity signal;
said primary polarity signal connected to said primary contact, said first secondary signal connected to said first secondary contact and said second secondary signal connected to said second secondary contact.

8. A cardiac electrode assembly according to claim 7 wherein said first and second polarity signals are energized out of phase to one another.

9. A cardiac electrode assembly according to claim 7 wherein said source of a pacing signal is a pulse generator connected to said contacts by electrical conductors.

10. A cardiac electrode assembly according to claim 7 wherein said source of a pacing signal is a pulse generator connected to said contacts by wireless transmission.

11. A method for treating a disease of the heart comprising:
placing a primary electrode in contact with a surface of the heart, said primary electrode having a first polarity when energized;
placing at least two secondary electrodes in contact with a surface of the heart, said secondary electrodes having a second polarity different from said first polarity when energized;
energizing said secondary electrodes at different times when energizing said primary electrode.

12. A method according to claim 11 wherein said electrodes are coupled to a controller by a wireless transmission.

13. A device for treating a condition of the heart, said device comprising:
a wall tension release device for said heart and including a material surrounding the heart and adapted to relieve wall tension on said heart;
an electrode assembly including:
a primary electrode in contact with a surface of the heart, said primary electrode having a first polarity when energized;
at least two secondary electrodes in contact with a surface of the heart, said secondary electrodes having a second polarity different from said first polarity when energized;
said primary and secondary electrodes adapted to be coupled to a controller for energizing said secondary electrodes at different times when energizing said primary electrode.

14. A device according to claim 13 wherein said electrodes are connected to leads extending from said wall tension release device.
